(19) United States
(12) Patent Application Publication
(10) Pub. No.: US 2013/0281646 A1
(43) Pub. Date: Oct. 24, 2013

(54) MULTIFUNCTIONAL LINKERS

(71) Applicants: KENNETH KORZEKWA, WAYNE, PA (US); MARK J. ROSEN, BRYN MAWR, PA (US)

(72) Inventors: KENNETH KORZEKWA, WAYNE, PA (US); MARK J. ROSEN, BRYN MAWR, PA (US)

(21) Appl. No.: 13/903,879

(22) Filed: May 28, 2013

Related U.S. Application Data
(63) Continuation of application No. 12/729,972, filed on Mar. 23, 2010, now abandoned.
(60) Provisional application No. 61/162,654, filed on Mar. 23, 2009.

(51) Int. Cl. A61K 47/48

(52) U.S. Cl. CPC A61K 47/4823; A61K 47/48176
USPC 526/264; 536/112

(57) ABSTRACT

The present invention relates generally to multifunctional polymeric linkers capable of linking a plurality of biologically active compounds. More particularly, the invention relates to the use of such multifunctional linkers that can effectively present two or more ligands simultaneously to two or more biological targets.

### FIELD OF THE INVENTION

The present invention relates generally to multifunctional polymeric linkers capable of linking a plurality of biologically active compounds. More particularly, the invention relates to the use of such multifunctional linkers that can effectively present two or more ligands simultaneously to two or more biological targets.

### BACKGROUND OF THE INVENTION

Scientists have sought effective linker molecules for linking biologically active agents when such agents are advantageously administered in tandem. Possible advantages of multifunctional compounds include increased combinations of affinity, activity, and selectivity.

Another advantage of multifunctional linkers is the ability to interact with multiple targets using only a single molecule. One of the most frequent causes of failure in a drug development program is the lack of efficacy in human trials. These failures are frequently not due to the failure to present a drug molecule to the target, but instead suggest that interaction at the receptor or enzyme is not sufficient for therapeutic effect. One possible reason that a target cannot be validated is the presence of redundant pathways (Hopkins, 2008). Synthetic lethality experiments suggest that redundant pathways buffer each other biologically (Ooi et al., 2006), and could prevent a sufficient response when only one pathway is targeted.

The presence of redundant or alternative biological pathways creates a dilemma for drug discovery. For many diseases, therapies comprising more than one drug are known in the art, and combination formulations are becoming more common. However, when a combination therapy is developed, often at least one of the drugs has been approved, and often both are already approved as single drugs. If compensatory pathways are present, it may be impossible to demonstrate sufficient efficacy with a single drug that binds to a single target. Without the approval of one of the components drugs, it is difficult to develop combination therapies.

One approach to this problem is to develop molecules with affinity to multiple targets. However, it is unlikely that a small drug molecule can be developed with sufficient affinity to multiple pharmacophores. The multifunctional polymeric linkers of the invention provide an alternative approach. Advantages of having multiple ligands on a single polymer backbone include: a single NCE is developed, instead of a combination therapy; the apparent affinity to receptors could be increased by interactions with multiple receptors; activity could be synergistically increased by interactions with multiple targets in the same area of cell surface membranes (local effects); and selectivity can be increased by interactions with multiple receptors on a cell type.

Systems have been reported in which multiple copies of the same ligand, or two different ligands are attached to a polymer matrix. In one study (Klutz, Gao, Lloyd, Shainberg & Jacobson, 2008), a polyamidoamine dendrimer was used to present multiple adenosine receptor agonists. Some of the compounds tested showed an increase in affinity and selectivity. However, the authors suggest that the data is not indicative of simultaneous binding to multiple receptors. In other studies, multiple carbohydrate ligands were found to increase the interaction with L-selectin (Gestwicki, Cairo, Strong, Oetjen & Kiessling, 2002; Kiessling, Gestwicki & Strong, 2006). Also, the importance of clustering of B-cell-antigen receptors in the immune response was shown with multiple-hapten polymers (Dintzis, Okajima, Middleton & Dintzis, 1990; Dintzis, Okajima, Middleton, Greene & Dintzis, 1989).

Early work by Porteguese used relatively small linkers to connect multiple opioid receptor agonists to a polyamide backbone (Portoghese, 2001; Portoghese et al., 1986; Portoghese, Ronsisvalle, Larson & Takemori, 1986). In these studies, the authors suggest that the similar distances for the observed optimum spacer length (22 Å, extended) and the receptor distance predicted for a homodimer (27 Å) supports that μ-opioid homodimers are present. However, a bivalent agonist with no spacer achieved similar enhancements in affinity for κ-opioid receptors.

Another study by Yano et al. looked at receptor interactions of two different ligands separated by sarcosine polymer linkers (Yano, Kimura & Imanishi, 1998). However, the data does not convincingly show simultaneous interactions with two different receptors, since similar enhancements were seen without a spacer.

In many other systems, small-molecule drugs bound to polymer matrices are designed as prodrugs (Lu, Shiah, Sakuma, Kopecková & Kopecek, 2002). Many of these efforts have been in cancer chemotherapy (Kopecek, Kopecková, Minko & Lu, 2000), but other therapeutic areas such as rheumatoid arthritis (Wang et al., 2007). For these compounds, the small molecule therapeutic is bound covalently to a polymer backbone using either a biodegradable linker or a biodegradable backbone. After reaching the target, the small drug molecules are released from the linker or polymer by chemical or enzymatic degradation. The result of polymer conjugation is either improved pharmacokinetics or uptake into tumors through the enhanced permeability and retention (EPR) effect (Maeda, Bharate & Daruwalla, 2008). Similar polymers have been reported that incorporate multiple different ligands, including targeting ligands or antibodies (David, Kopecková, Minko, Rubinstein & Kopecek, 2004; Luo, Bernshaw, Lu, Kopecek & Prestwich, 2002; Pan et al., 2008) and multidrug therapies such as incorporating an antiestrogen and a cytotoxic agent into a chemotherapeutic (Greco et al., 2007). It should be noted that release of the therapeutic is thought to be necessary for these compounds to be active (Malugin, Kopecková & Kopecek, 2007).

These examples of multifunctional polymers may not be useful as a general method to simultaneously present multiple ligands to multiple targets. In general, the distances of the polymer linkers used are either too short, too hydrophobic, or are not sufficiently flexible to simultaneously interact with multiple ligands.

US Patent Application 2004/0023290 discloses, among other things, a system designed to present multiple ligands to multiple targets with various linkers. However, the linkers described therein are relatively short for the distances that may need to be covered for effective multiple presentation of distinct ligands. For example, the preferred linkers therein are said to provide a “minimal, shortest path distance between adjacent ligand groups [that] does not exceed 100 atoms or 40 angstroms.”

Previous multifunctional polymers are short because they confer a broader probability distribution for the end-to-end distances, and tend to avoid non-specific interactions between the ligands and cellular components, as found in longer molecules. The art is in need of multifunctional linkers capable of presenting a plurality of ligands simultaneously at distances required for efficacy. The present invention provides a solution to the problems of presenting multiple ligands at distances that can exceed 40 angstroms.

### SUMMARY OF THE INVENTION

It is an object of the invention to overcome the drawbacks of prior linkers and methods of using same. The present invention provides a method by which distal linkers that present ligands to targets are separated by a rigid scaffold. This method overcomes the deficiencies inherent in long linker length when presenting multiple ligands simultaneously to multiple targets.

In one aspect, the invention provides a multifunctional linker capable of binding a plurality of ligands, having at least one central portion and at least two distal portions, wherein the distal portions are capable of binding ligands. In one aspect, the central and distal portions comprise a polymer. Suitable polymers may be selected from polysaccharides, hyaluronic acid, PVP, synthetic polymers, and derivatives thereof, as well as other useful polymers.

In another aspect, the central portion is rigid and the distal portions are flexible, or alternatively, the central portion is flexible and the distal portions are rigid. The central and distal portions are rigid or flexible according to the design of the linker in order to achieve a particular distances between bound ligands and persistence lengths of the linkers.

Thus, in one aspect, the distal portions are rigid and separated from the central portion with flexible hinge regions.

In another aspect, the multifunctional linker provides a plurality of distal portions spaced at desired intervals about the central portion, providing a higher degree of granularity of control over the distances between the ligands.

The ligands may be the same or different biologically active agents, targeted to a variety of receptors. In another aspect, pharmaceutical compositions are provided which use the multifunctional linker with bound ligands as a medicament.

These and other objects are achieved through the present invention as exemplified and further described in the Detailed Description of the Invention below.

### BRIEF DESCRIPTION OF THE DRAWINGS

FIG. 1 is a schematic representation of an embodiment of the invention in which a linker has a rigid central section and flexible distal sections.

FIG. 2 is a schematic representation of an embodiment of the invention in which a linker has a rigid central section and rigid distal sections connected by flexible hinges.

FIG. 3 is a schematic representation of an embodiment of the invention in which a linker has a large, branched, rigid central section, and multiple flexible distal sections.

FIG. 4 is a schematic representation of an embodiment of the invention in which a linker has a large, branched, rigid central section, and multiple rigid distal sections connected with flexible hinges.

FIG. 5 is a schematic representation of an embodiment of the invention in which a linker comprises a cellulose-PVP polymer with ligands capable of simultaneously binding to two receptors, one of which is a homodimer.

### DETAILED DESCRIPTION OF THE INVENTION

The multifunctional linkers provided by the invention are multifunctional scaffold molecules, generally but not limited to polymers, which are capable of having covalently bound ligands for simultaneous presentation to multiple targets. The multifunctional linker may remain intact as its ligands interact with multiple receptors. These receptors can be associated with any of a variety of pathways and associated targets, for example:
Redundant pathways for which interacting with multiple pathways results in increased efficacy
Synergistic pathways for which efficacy is achieved with lower doses
Multiple pathways that decrease the possibility of resistance
Decreased toxicity due to increased selectivity for the desired therapeutic response
Targets for which approved drugs are not available, allowing the more facile approval of a single drug which targets multiple pathways
When the targets are present in the same membrane, simultaneous binding to multiple targets can increase the effective affinity of both ligands, increasing efficacy.

Receptors targeted by the ligands may include any of a variety of cellular receptors, enzymes, and any other cellular or tissue component that results in targeting of the multifunctional linker, or upon binding of the multifunctional linker results in a biological response.

Ligands are generally biologically active agents, such as pharmaceutical compounds, prodrugs, small molecules, peptides, peptidomimetics, and the like. By way of nonlimiting examples, ligands may include:
Angiogenesis inhibitor ligands including VEGF inhibitors, PDGF inhibitors, integrin inhibitors, thrombospondin antagonists, TGFb inhibitors, somatostatin analogs, CXCR4 inhibitors, herceptin agonists, ANG II antagonists, galectin-1 inhibitor, decorin LRR5 peptides, and angiostatin analogs.
Antiproliferative ligands including somatostatin analogs, MGSA/GROa peptide, Herceptin analogs EGFR inhibitors, bombesin/GRP antagonists, TRAIL agonists, ANG II antagonists, pro-apoptotic marine peptides, α-fetoprotein inhibitors, and TGFβ inhibitors.
Ligands to prevent stromal activation including TGFβ inhibitors, PDGF inhibitors, VEGF inhibitors, MMP inhibitors, integrin inhibitors, uPA peptides, thrombospondin analogs, E-selectin analogs, and fibroblast-activation protein-α analogs.
Ligands to prevent fibrosis including TGF-β1 inhibitors, TNF-α inhibitors, IL-6 inhibitors, IL-3 inhibitors, endothelin-1 inhibitors, IGF-1 inhibitors, neutrophil elastase inhibitors angiotensin II inhibitors, integrin inhibitors, PAR1 inhibitors, thrombospondin inhibitors, and thrombin inhibitors.
Ligands to prevent inflammation including TNF-alpha inhibitors, IL-1 inhibitors, IL-6 inhibitors, IL-12 inhibitors, IL-15 inhibitors, IL-17 inhibitors, IL-23 inhibitors, Adenosine A3 agonists, CD20 antagonists, CD8 antagonists, TLR antagonists, TGFβ agonists, L-selectin inhibitors, E-selectin inhibitors, and integrin inhibitors.

Those of skill in the art will appreciate that many other suitable ligands are amenable to use in the present invention.

In order to provide a scaffold to simultaneously present multiple ligands to multiple receptors, the polymer backbone should allow free “solution-like” characteristics for the covalently attached ligands. Therefore, the useful polymers are likely to be hydrophilic and water soluble. Hydrophobic polymers are likely to interact with membranes and interact with the ligands themselves. Even the ampiphillic polyethylene glycol (PEG) molecules are moderately hydrophobic (Hammes & Schimmel, 1967). Covalently linked PEG molecules are thought to interact with hydrophobic portions of protein molecules and protect them from enzymatic degradation, presumably by shielding the peptide from proteolytic enzymes (Caliceti & Veronese, 2003). Endothelial cells and erythrocytes can also take up PEG-protein conjugates (Bhat & Timasheff, 1992), suggesting that there are interactions with membranes.

In one embodiment, the polymer scaffold uses polyvinylpyrrolidone (PVP) (Haaf, Sanner & Straub, 1985). PVP is inert and is readily excreted, and is used as a plasma expander, a disintegrant, and a food additive. The ability to achieve significant distances between the covalently bound ligands will be necessary to simultaneously interact with multiple receptors (Jeppesen et al., 2001). PVP can achieve the same hydrodynamic radius with fewer backbone atoms than PEG (Armstrong, Wenby, Meiselman & Fisher, 2004), suggesting that the solution conformation of PVP is more extended than PEG. PVP conjugates, when compared to PEG and dextran conjugates were shown to have the lowest volume of distribution, suggesting that they have a lower propensity to enter cells (Kaneda et al., 2004). Provided that the length of the polymer backbone is sufficient, PVP, PVP copolymers, or PVP linkers could be used to present multiple various ligands to multiple receptors.

The branched nature of the polymer and the incorporation of linkable copolymer components are important considerations in the polymer carrier to be used. Terminally conjugated linear homopolymers can have two ligands per polymer molecule, if both ends of a linear polymer are available for conjugation. Branched and multifunctional linear polymers and copolymers offer the possibility of multiple ligands per polymer carrier molecule. Dendrimers are an interesting class of highly branched polymers (Lee, MacKay, Fréchet & Szoka, 2005). The larger the dendrimer molecule, the more sites can be functionalized. Functionalized dendrimers have been used to present multiple adenosine receptor agonists to cell surface receptors (Klutz et al., 2008).

There is a considerable problem of statistics that must be addressed when considering linear-flexible homopolymers in solution. For example, in the case of homo- or hetero-dimer receptors, the distance between them is generally in the range of between 25-50 Å (Livnah et al., 1999; Portoghese, 2001). A multifunctional linker of the invention is thus constructed which presents ligands at the desired distance by providing its effective length in the desired range, notwithstanding its fully extended length.

As another example, in order to simultaneously bind to two enzymes or receptors, the multifunctional linker must be long enough to present ligands at both binding sites. If both independent targets are present at a membrane surface, it can be expected that distances of 50-100 Å will be required, though that distance may be as large as 500 Å (Livnah et al., 1999). As with the dimer target, a multifunctional linker is constructed whose effect length matches the required distance between the receptors.

For example, consider the case of a pair of independent receptors which are 75 Å apart. For PVP, the monomer length is about 2.2 Å. Therefore, a polymer of N=35 can reach 75 Å if fully extended. However, the N=35 polymer is far more likely to exist in a partially folded state, bringing the functionalized ends of the polymer closer together and decreasing the effective length of the polymer. Indeed, the probability of the polymer existing in its fully extended conformation approaches zero.

A useful parameter to describe the end-to-end effective length of a polymer is the Flory radius R_{F}, a parameter describing the generally spherical three dimensional structure of the folded polymer. For a PVP polymer of N=100, R_{F}=35 Å, considerably less than the desirable distance of between 50 and 100 Å, preferably about 75 Å, for presenting ligands to two independent receptors. An R_{F} of 75 Å will require a PVP polymer in the range of N=360. Using the Gaussian distribution function and the Flory radius to represent the average distance between monomers, the probability distribution for a random coil polymer is represented by Equation 1.

$$p ( R ) = 4 π {{R}^{2} ( \frac{2 π}{3} 〈 {r}^{2} 〉 )}^{- \frac{3}{2}} {e}^{- ( \frac{3}{2} \frac{{R}^{2}}{〈 {r}^{2} 〉} )} \qquad Equation 1$$

where <r²>^{0.5} equals the Flory radius. Therefore, the probability of achieving a distance of 75 Å for a polymer with a Flory radius of 35 Å is ˜50× lower than achieving a distance of 35 Å. However, if the polymer length is increased, the overall distribution of the end-to-end length (that is, the distribution of all possible lengths and the frequency or likelihood of existing at that length) is broader. Thus, increasing chain length does increase the average end-to-end effective length, but a penalty is paid with a broader distribution of various lengths of the polymer. This suggests that it may be difficult to use linear homopolymers to achieve 75 Å distances without sacrificing affinity. Also, from a practical perspective, larger polymers tend to increase the cost-of-goods and can cause formulation difficulties.

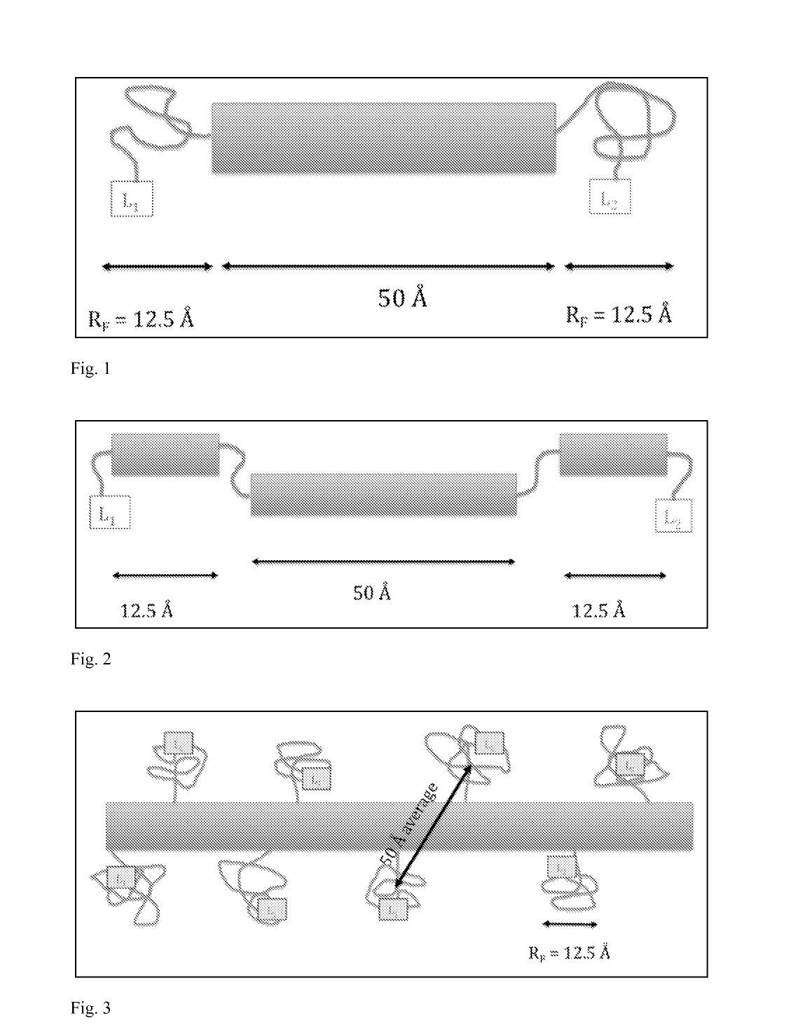

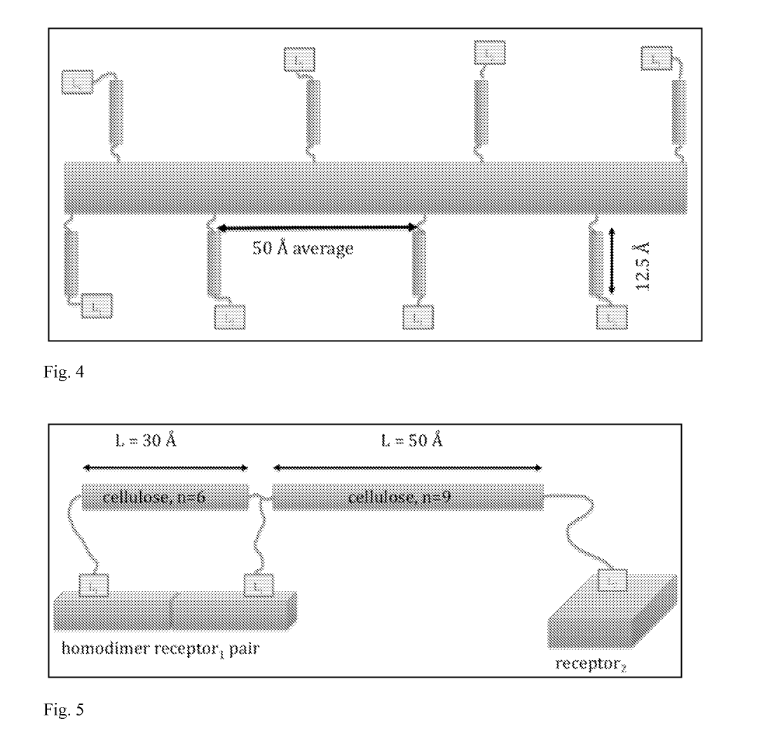

The invention provides polymer configurations which avoid these penalties by using rigid portions that allow designing of the multifunctional linkers to have effective lengths matching the desired distance between the target receptors, and thereby achieving simultaneous interactions therewith. In general, these polymers can be (1) linear copolymers with a rigid central section and flexible distal sections; (2) linear copolymers with a rigid central section and rigid distal sections linked to the central section by a flexible linker; (3) branched copolymers with a large rigid central section and multiple flexible distal sections; or (4) branched copolymers with a large rigid central section and multiple rigid distal sections linked to the central section by flexible linkers. These preferred configurations are shown schematically in FIGS. 1-4. These multifunctional linkers are optimally designed to present multiple ligands to multiple targets at appropriate distances.

Such distances may be in the range of 25-500 Å; for the case of dimers, the distances are in the range of about 25-50 Å, while for the case of independent receptors, the distances may range from about 40-500 Å, more preferably 40-400, more preferably 40-300, more preferably 40-200, more preferably 40-100 Å.

The multifunctional linkers depicted in FIGS. 1-4 employ a rigid central section to separate distal sections to which the ligands are attached. This permits the average distance between the ends to be increased by the length of the rigid section of polymer. If a 50 Å rigid section of polymer has 20-mer sections of PVP at each end (R_{F}=12.5 Å each), the effective end-to-end length of the linker would be 75 Å, a match for the desired distance between the example of target receptors discussed above. More importantly, the length distribution for the second ligand that binds would be six-fold narrower than the N=360 polymer (the number of monomers required to achieve the 75 Å without such a rigid section) and the overall degrees of freedom would be greatly reduced. That is, the multifunctional linker with a central rigid portion would be far more likely to exist at the 75 Å desired length, and achieve the targeting of the two receptors, than a purely flexible N=360 polymer. Based on the surface areas of the two 12.5 Å spheres relative to one 75 Å sphere, an 18× decrease in radial distribution of the PVP chains is achieved, providing for better matching of the target distance, and a superior multifunctional linker. Thus, the rigid sections of the linkers confer finer control of the design of such linkers, adapted for the simultaneous presentation of ligands to a plurality of targets.

In one embodiment, the rigid section is constructed from a chain of a rigid polysaccharide with PVP chains on each end. Dextran (formula I) and cellulose (formula II) are polysaccharides that are semi-rigid and rigid, respectively, and both are particularly amenable for use in the invention.

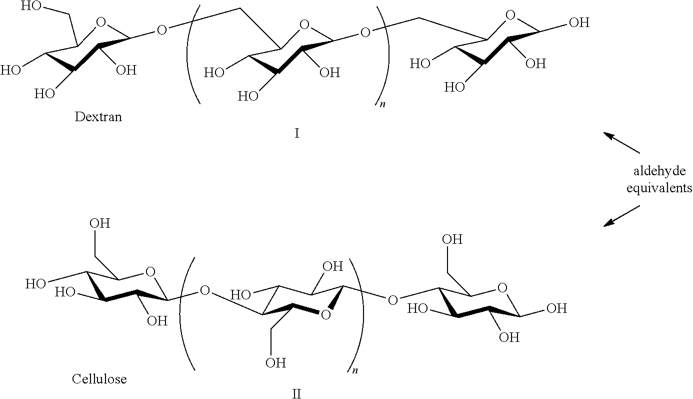

Dextran has a persistence length (Lₚ) reported to be 15-30 Å. The persistence length is the distance at which the correlation between the directional tangent and length is lost, and is therefore a measure of rigidity. Although very large dextran molecules are considered to be flexible, for distances that are required for multifunctional linkers (about 25 Å and greater), dextrans would be considered semi-rigid, since the persistence length approaches the extended length of the polymer. Small dextran spacers have been used for protein immobilization to solid supports (Penzol, Armisén, Fernández-Lafuente, Rodés & Guisán, 1998). Dextran is particularly water-soluble and non-toxic. Reactions for the selective functionalization of both the aldehyde and primary alcohol ends of dextran have been reported. Since commercial dextran is about 5% branched (one or two glucose monomers per branch), a 2000 molecular weight dextran would be primarily unbranched. Dextran-PVP polymers are shown in formulas III and IV below.

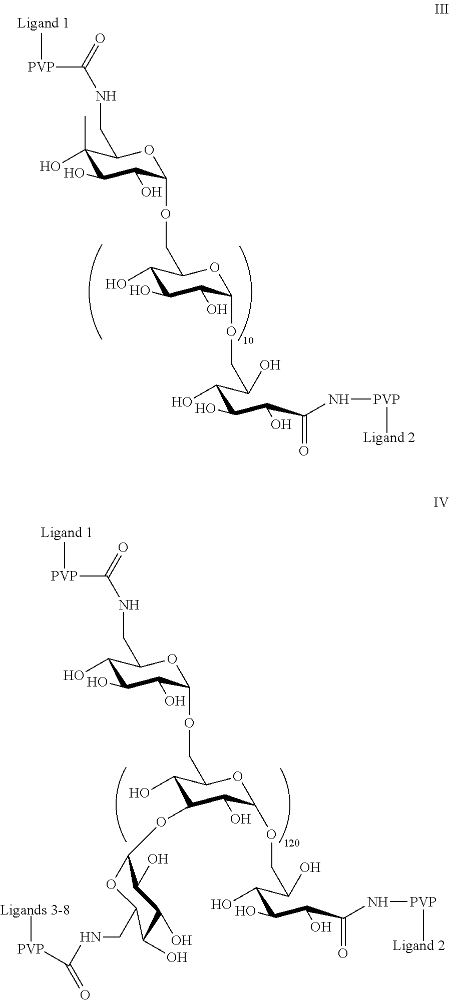

In addition to serving as linear spacers, larger polysaccharides could also be used as rigid central regions for multifunctional polymers. For example, larger dextran molecules behave as ellipsoids in solution with a more extended conformation than random polymers (Bohrer, Deen, Robertson, Troy & Brenner, 1979). A 20,000 MW dextran has a major to minor axes ratio of 9, having dimensions of approximately 20 Å by 180 Å. This corresponds to a folded bundle of four 180 Å dextran chains. This larger central dextran backbone is branched (1/20 residues), and is useful for multifunctionalization. A 20,000 MW dextran polymer has, on average, eight primary alcohol sites which can be conjugated to the flexible portion of the linker. Assuming the branches are randomly distributed on the surface of this ellipsoid, an average distance between PVP chains of ˜40 Å would be expected. Thus, the natural branches of dextran are ideally suited for use as a rigid polymer for multifunctionalization, as seen in formula III (linear dextran-PVP multifunctional polymer) and formula IV (branched dextran-PVP multifunctional polymer).

In addition to the natural branches, the secondary alcohols can be partially modified to provide any number of flexible linkers per dextran molecule. The persistence length of dextran (15-30 Å) suggests that a short central dextran section will be mostly unfolded.

If a more rigid polysaccharide is desired, a cellulose polymer can be used (formula II). With a persistence length of 150 Å, a 50 Å spacer of a cellulose derivative such as carboxymethyl cellulose is quite rigid. Cellulose polymers up to 100 Å in length have been recently synthesized by a cationic ring polymerization reaction. Like dextran, cellulose is also non-toxic and hypoallergenic. Since linear cellulose molecules can be synthesized by polymerization of protected monomers (Nakatsubo, Kamitakahara & Hori, 1996), the reducing sugar and initiating alcohol can be derivatized for functionalization. The added control provided by a synthetic polysaccharide could be used to fine tune a multifunctional polymer. For example, in one embodiment, a multifunctional polymer is designed to interact with two receptors, where one is a homodimer receptor, and the second, another independent receptor (or even another copy of the same receptor or dimer receptor). The synthetic procedure of the invention permits a multifunctional polymer to be designed with shorter cellulose spacers (e.g. 30 Å, n=6) between L₁ pairs and longer spacers (50 Å, n=9) between L₁ and L₂ pairs. For example, one polymer with this configuration is a linear polymer with the structure L₁-PVP]-[30 Å spacer]-[L₁-PVP]-[50 Å spacer]-[L₂-PVP] (see FIG. 5 and example 6). In this embodiment, fine control is conferred upon the multifunctional linker, providing it with the ability to bind both to the homodimer with shorter distances between them, and the second receptor, a greater distance from the dimer.

Another attractive rigid or rod-like polymer is polyproline. As early as 1973, polyproline was used as a rigid spacer for hapten presentation (Ungar-Waron, Gurari, Hurwitz & Sela, 1973). In aqueous solution, polyproline forms a rigid PP Type II left-handed helix (Schuler, Lipman, Steinbach, Kumke & Eaton, 2005). Also, polyproline dendrimers have been prepared using either 4-aminoproline or the imidazole analog as a branch point (Crespo et al., 2002; Sanclimens, Crespo, Giralt, Royo & Albericio, 2004). Examples of polyproline-PVP multifunctional polymers are shown in formula V and formula VI below. Advantages of polyproline include: (1) facile synthesis using standard peptide synthesis techniques, (2) a homogeneous central rigid section, and (3) diverse protection schemes can be used that allow exact functionalization of each derivatization site.

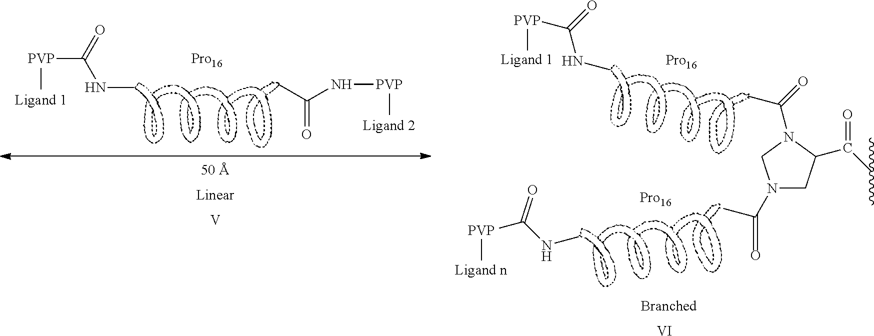

An additional advantage of incorporating a polyproline helix is the potential to make the compound cell-penetrating by modification of the proline residues (Geisler & Chmielewski, 2007; Yoon, Lim, Lee & Lee, 2008) (see discussion on cell penetrating peptides below). Formula V provides an example of a polyproline-PVP bifunctional polymer, while formula VI provides an example of a polyproline-PVP multifunctional polymer.

In other embodiments, rigid polymers may be hydrophilic and have effective persistence lengths of >10 Å. These may be biopolymers, such as peptides with significant secondary structure (helices or sheets), or rigid synthetic polymers. Many rigid synthetic polymers have been reported that are useful as rigid linkers including polyamides resulting from condensation of aromatic diamines and aromatic dicarboxylates, polydiacetylenes, etc. However, many of these polymers will be inherently hydrophobic unless hydrophilic substituents are introduced onto the backbone. In addition, there will be significantly more risk in using polymers that have not seen significant clinical use.

If more than two ligands are to be bound to the same polymer molecule, a branch point will be required. Although multiligand polymers have been prepared by functionalizing a linear backbone (Lee & Sampson, 2006), optimum flexibility is achieved with a branched polymer. Again. Inclusion of rigid sections on each branch allows for a more optimal end-to-end distance.

Since water-soluble polymer backbones are most effective for presentation of multiple ligands to multiple receptors, these therapeutics based on the multifunctional linkers of the invention may be restricted to extracellular targets, unless they are modified for cell penetration. Cell-penetrating peptides have been developed to facilitate the translocation of large molecules and liposomes to intracellular spaces. These include the penetratins (Langel, 2006), TAT-peptides (Torchilin, Rammohan, Weissig & Levchenko, 2001), and other basic, ampipathic peptides (Deshayes, Morris, Divita & Heitz, 2006; Maier et al., 2006; Rhee & Davis, 2006; Schröder et al., 2008). A TAT-peptide has been used to deliver an HPMA copolyler-doxodubicin into the cytoplasm and nucleus of ovarian cancer cells (Nori, Jensen, Tijerina, Kopecková & Kopecek, 2003). Thus, intracellular targets may be approached by adding a cell-penetrating peptide (or other means to achieve cell-penetration) to the multifunctional linker therapeutics. An example has been reported in which polyproline helices were modified by putting basic substituents on proline residues at appropriate positions on the helix (Fillon, Anderson & Chmielewski, 2005). This resulted in an amphiphilic helix which could be use for cell penetration. Thus, the rigid linker can be modified to increase cell penetration of a multifunctional polymer therapeutic.

Many technologies are available for the construction of the polymer-ligand configurations described above. One of the more attractive approaches for construction of these multifunctional polymers is to prepare rigid and flexible building blocks which can be combined to form the polymer scaffold. There are many reactions that have been used to prepare polymer conjugates (Gauthier & Klok, 2008). One of the more useful reactions that have been used recently is the alkyne-azide cycloaddition reaction. With this reaction, polymer and ligand building blocks are functionalized with either alkyne or azide groups. These groups are then ligated in the presence of a copper catalyst under very mild conditions. The selectivity of this reaction provides great flexibility and efficiency in combining mixtures of various polymers and ligands. Use of this reaction allows the ligands to be ligated to various flexible sections, which can be combined with different rigid polymers to optimize activity.

With the preceding disclosure of the invention, those of skill in the art will readily appreciate the many uses of the multifunctional linkers described. The linkers are capable of binding ligands at their at least two ligand binding sites. The ligands may be the same, where presentation of multiple copies of the same ligand is desired, or may be different, for simultaneous presentation of multiple ligands to multiple receptors.

Therapeutics may be constructed using the multifunctional linkers of the invention, by binding ligand to the ligand binding sites of the linkers. Such therapeutics may thereby achieve increased efficacy due to the simultaneous presentation of multiple ligands, targeting multiple receptors, or even multiple presentation of the same ligand targeting multiple copies of the target receptors. Such therapeutics may be formulated as pharmaceutical compositions, and may comprise additional pharmaceutically-acceptable carriers, excipients, and the like.

Other embodiments, uses, and advantages of the present invention will be apparent to those skilled in the art from consideration of the specification and practice of the invention disclosed herein. The specification and examples should be considered exemplary only. The intended scope of the invention is only limited by the claims appended hereto.

### EXAMPLES

The present invention will be further understood by reference to the following non-limiting examples.

### Example 1

### Synthesis of a Flexible Polymer-Ligand Building Block with an Alkyne Linker

A PVP of length 20 monomer units attached to a ligand is prepared using the ATRP polymerization procedure (Lutz, Borner & Weichenhan, 2006). The synthetic procedure is shown in Scheme 1. Briefly, vinylpyrrolidone polymerization can be initiated by the addition of an a-bromoester, in this example, bromoisobutyric acid ethyl ester. The ratio of monomer to initiator will determine the length of the resultant polymer; in this example, a ratio of about 20 to 1 is appropriate. After consumption of the monomer, the bromo-terminated polymer is reacted with propargylamine to give the terminal alkyne. Hydrolysis of the ester and activation with N-hydroxysuccinamide allows for the nucleophilic addition of the free amine on a ligand. This building block, consisting of a ligand covalently bound to a flexible polymer with a terminal alkyne can be attached to a rigid central polymer derivatized with an azide group.

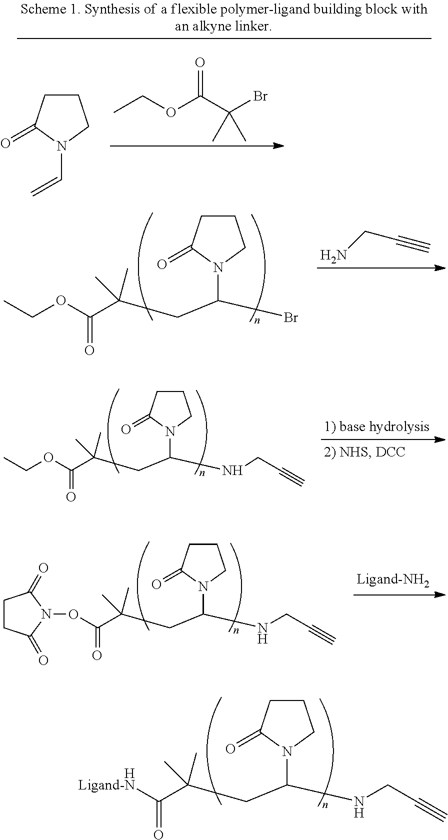

### Example 2

### Synthesis of a Flexible Polymer-Ligand Building Block with an Azide Linker

A 30-mer length of PVP attached to a ligand can be prepared using the ATRP polymerizaion procedure. The synthetic procedure is shown in Scheme 2. Briefly, vinylpyrrolidone polymerization can be initiated by the addition of an a-bromoester. The ratio of initiator to polymer will determine the length of the resultant polymer. After consumption of the monomer, the bromo-terminated polymer is reacted with sodium azide to produce the terminal azide. Hydrolysis of the ester with Me₃SnOH (Nicolaou, Estrada, Zak, Lee & Safina, 2005) and activation with N-hydroxysuccinamide allows for the nucleophilic addition of the free amine on a ligand. This building block, consisting of a ligand covalently bound to a flexible polymer with a terminal azide can be attached to a rigid polymer derivatized with an alkyne group.

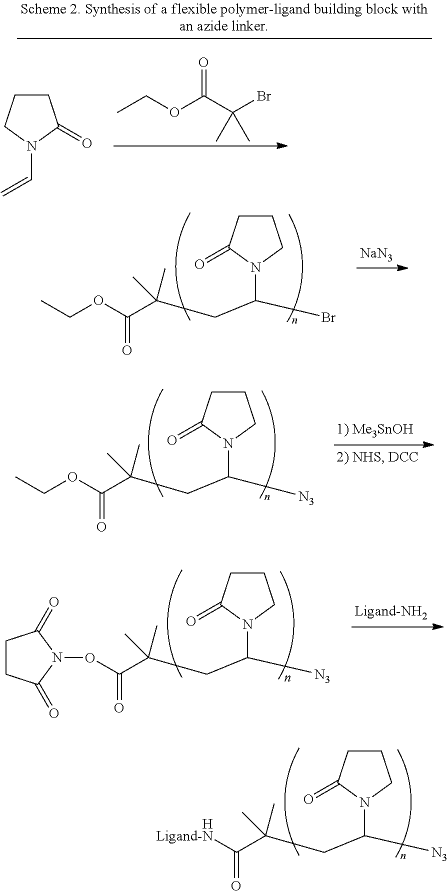

### Example 3

### Preparation of Azide-Derivatized or Alkyne Derivatized 20,000 MW Dextran

The aldehyde and primary alcohols of dextran are converted selectively to carboxylic acids by treatment with 4-acetamido TEMPO and 2 equivalents of potassium peroxomonosulfate per primary alcohol plus ½ equivalent per aldehyde. For a 20000 MW dextran, this results in eight carboxylates per molecule. The azide derivative are prepared by converting the carboxcylic acid groups to the activated ester with N-hydroxysuccinamide. Addition of azidoethylamine to the activated ester gives the dextran an azide group. If the central linker must be attached to a rigid distal polymer, azidopropylamine may be used to provide more flexibility. To convert the carboxylate to the terminal alkyne, the activated ester is derivatized with 1,1-dimethyl-2-propynylamine.

Alternatively, the secondary alcohols of dextran are partially converted to carboxymethylate dextran. Iodoacetic acid (1.7% based on total alcohol content) is added to dextran (MW 20000) to provide ligation sites to 5% of the glucose monomers. The carboxylate is converted to the activated ester with N-hydroxysuccinamide. Addition of azidoethylamine to the activated ester gives the azide-derivatized dextran. A number of non-specific alkynylation reactions are possible including the reaction of dextran with 4-bromobutyne in the presence of base (Example 10).

### Example 4

### PVP-Polyproline Bifunctional Polymer

In this Example, the flexible polymer-ligand building blocks of Example 1 are used to construct a PVP-polyproline multifunctional polymer. The reaction is shown in Scheme 3. The primary amine of azidopropylamine is linked to a PAL-aldehyde resin by reductive amination. Solid phase peptide synthesis (SPPS) is conducted on the resulting secondary amine. After adding the required number of proline residues, an azido pentanoic acid is added to the N-terminus of the last proline residue. The first ligand-PVP-alkyne building block is added to the azide by the Cu(I) catalyzed alkyne-azide [2+3]cycloaddition reaction (Kolb, Finn & Sharpless, 2001). The peptide-PVP-Ligand conjugate is cleaved from the resin and the second ligand-PVP-alkyne building block is added to the C-terminal azide by the Cu(I) catalyzed alkyne-azide [2+3]cycloaddition reaction.

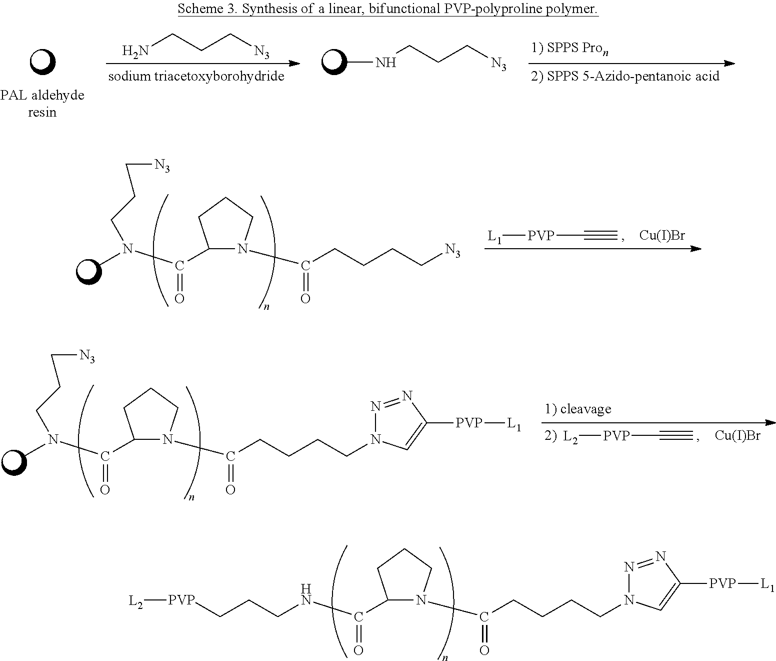

### Example 5

### Synthesis of Short Linear Dextran Building Blocks

The method to synthesize short, linear dextran polymers derivatized with an amino and carboxylate termini is shown in Scheme 4. The reducing sugar terminus of the dextran molecule, preferably n≦10, is converted to the Boc-protected amine as described previously as described previously (Goodwin et al., 2009) with the mono-Boc protected 2-aminoethanol, DCC and NHS. This reaction selectively converts the acetal form of the terminal aldehyde to an ether. The primary alcohol terminus is converted to the carboxylate by oxidation with 4-acetamide-TEMPO and 2 equivalents of potassium peroxomonosulfate.

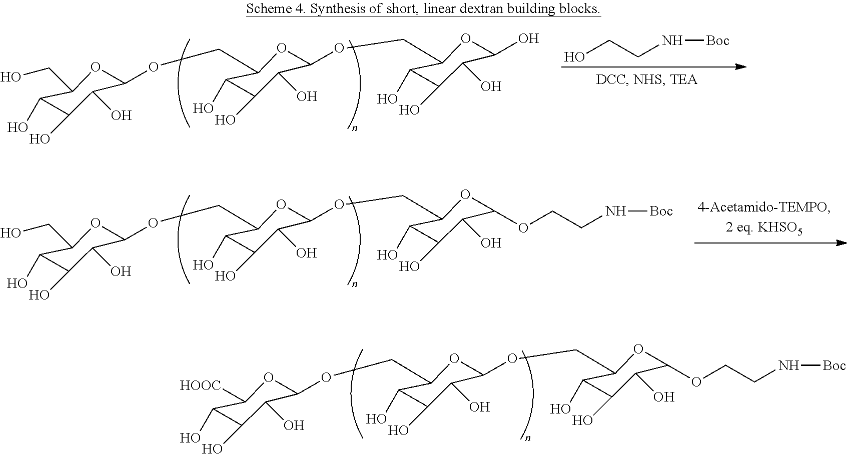

This intermediate can be used to provide for a rigid central dextran linker or a rigid distal linker. To provide a central linker, the carboxylic acid and can be converted to a terminal azide by converting the carboxcylic acid to the activated ester with N-hydroxysuccinamide. Addition of azidoethylamine to the activated ester gives the terminal azide. If the central linker must be attached to a rigid distal polymer, azidopropylamine can be used to provide more flexibility. To covert the carboxylate to the terminal alkyne, the activated ester can be derivatized with 1,1-dimethyl-2-propynylamine. To convert the terminal amine to an azide, the deprotected amine is coupled with α-azidoisobutyric acid. To convert the terminal amine to an alkyne, the deprotected amine is coupled with 4-pentynoic acid.

If bound with a rigid distal linker, the ligand can be coupled with the terminal carboxylate as in examples 1 and 2. Coupling the deprotected amino terminus to 4-pentynoic acid provides a flexible linker to the rigid central section.

### Example 6

### Construction of a Trifunctional Polymer that Binds to a Homodimer and an Independent Receptor

For this example, carboxymethyl cellulose spacers are used to separate PVP flexible spacers that bind ligands L1 and L2. L1 binds to a homodimer with an optimum distance of 40 Å between binding sites and L2 binds another receptor with an optimum distance of 75 Å between receptors the L1 and L2 receptors. The synthetic scheme to prepare building blocks for a trifunctional polymer with the configuration [L1-PVP]-[30 Å spacer]-[L1-PVP]-[50 Å spacer]-[L2-PVP] is shown in Scheme 5. Briefly, The protected cellulose monomers are polymerized as reported (Nakatsubo et al., 1996) to obtain the desired degree of polymerization (6 or 9). A convergent synthesis method could also be used to synthesize building blocks of n=8 or less (Nishimura & Nakatsubo, 1996a; Nishimura & Nakatsubo, 1996b). The aldehyde terminus is converted to the Boc-protected amine as described previously (Goodwin et al., 2009) with the mono-Boc protected 2-aminoethanol, DCC and NHS. The terminal 4-hydroxy group is converted to the alkyne with N-methyl-propargylamine. After protecting the alkyne with a TMS protecting group, the benzyl groups are removed by Pd/H₂ reduction, and some of the available hydroxy groups are converted to the carboxymethyl ethers with chloroacetic acid. Standard degree of carboxymethyl ether incorporation is 0.6-0.9 carboxymethyl ether per glucose monomer. The ligand-PVP-alkyne building blocks described in Scheme 2 can be incorporated with a standard alkyne-azide cycloaddition reaction. These building blocks (L₁, n=6 and L₂, n=9) can be combined with a trifunctional linker to form [L₁-PVP]-[30 Å spacer]-[L₁-PVP]-[50 Å spacer]-[L₂-PVP]. One example is the use of Z-Glu-OBzl in which the [L₁-PVP]-[30 Å spacer] is bound to the unprotected carboxylate, followed by deprotection and propargylation of the amine, addition of the L₁-PVP-azide (Scheme 2), and finally addition of the [50 Å spacer]-[L₂-PVP] to the last carboxylate. These building blocks and similar building blocks of various sizes and configurations can be used to optimize binding to any combination of receptor configurations.

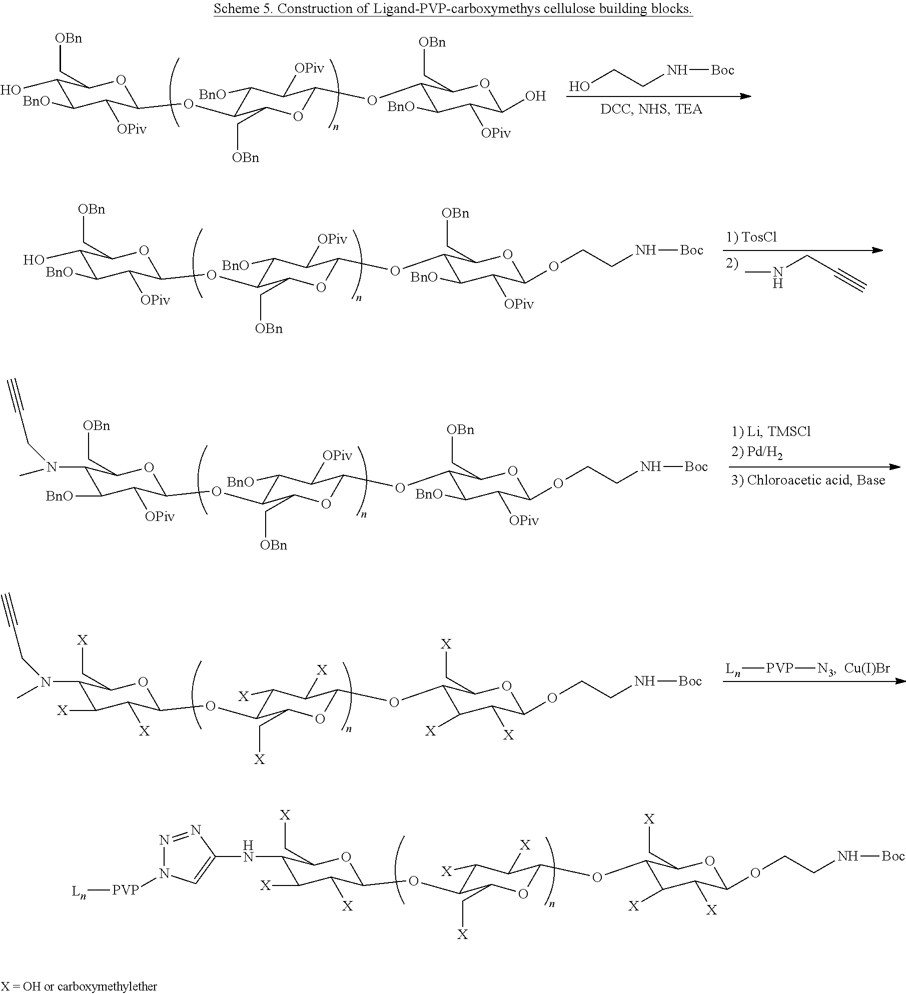

### Example 7

### Construction of a Therapeutic—a Bifunctional Angiogenesis Inhibitor

Angiogenesis inhibitors have been used to suppress tumor growth (Cao, 2008). Two targets for angiogenesis are the VEGF receptor and αvβ3 integrin. Inhibitors of each have been developed and have been shown to have moderate efficacy as individual therapeutics (Collinson, Hall, Perren & Jayson, 2008). Both VEGFR and αvβ3 integrin are present on the same endothelial cells and have been reported to interact synergistically (Hodivala-Dilke, 2008; Weis et al., 2007). Therefore, a soluble, multifunctional polymer containing a VEGFR inhibitor and an αvβ3 integrin inhibitor could show improved efficacy.

An example of such a synthetically therapeutic is a multifunctional polyproline-PVP molecule with both a VEGFR inhibitor and an αvβ3 integrin inhibitor (shown in formula VII below). For this example, the VEGFR inhibitor cyclo-VEGi (Ryu & McLamon, 2008; Zilberberg et al., 2003) and the αvβ3 integrin inhibitor Cilengitide (D'Andrea, Del Gatto, Pedone & Benedetti, 2006; Reardon, Nabors, Stupp & Mikkelsen, 2008) are used as ligands. Both are cyclic peptides of 17 and 5 amino acids, respectively. Cyclo-VEGF is based an a part of the VEGF sequence and cilengitide is an RGD peptide, c(-RGDf[NMe]V-).

Methods for conjugating Cyclo-VEGi with PEG linkers have been reported (Gonçalves et al., 2005). Since the PEG conjugates retain their activity, this provides a validated method for conjugating this molecule to the PVP distal sections. Cilengitide can also be conjugated, e.g. by substituting a D-tyrosine for the D-phenalanine, and conjugating to the phenol group. Other conjugated RGD peptides have been reported in the literature and are shown to be active (Dijkgraaf et al., 2007; Smolarczyk et al., 2006). Standard peptide protecting groups and synthetic methods can be used for preparation and conjugation of these ligands (Sewald & Jakubke, 2002).

A (Pro)16 spacer separates two 20-mer PVP chains by 50 Å, providing an average effective distance between ligands of 75 Å. This results in faster and therefore tighter binding. Formula VII depicts an example of such a linear, bifunctional polyproline-PVP therapeutic.

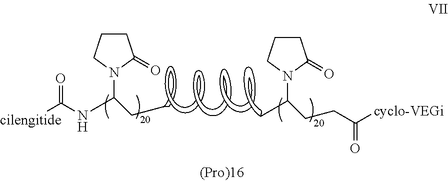

Similar molecules may be constructed with short, bifunctional or long multifunctional dextran molecules or other rigid linkers as the central rigid section. Likewise, the flexible distal sections may be replaced with rigid distal sections attached to the central rigid section with a flexible linker.

### Example 8

### Optimization of a Multifunctional Linker of the Invention

Optimization may be carried out on a convenient, cell-based system. One of the largest and most studied family of receptors is the GPCR receptor family. These are extracellular receptors that cause signal transduction through the use of second messengers including cAPM and Ca²⁺. The GPCRs are an ideal system for POC since 1) cell based assays are available for a large number of GPCRs, 2) many of these receptors have commercially available agonists, and 3) receptors can be chosen to give two different measurable responses. The two receptors-agonist pairs that have been chosen for this example are the A2-adenosine receptor and the agonist ADAR (Klutz et al., 2008) and the neurotensin receptor and a pentapeptide agonist (Yano et al., 1998). Both of these agonists have been used as polymer conjugates, removing an important uncertainty from the project. Also, the A2-adenosine receptor is gives primarily a Ca²⁺ signal whereas the neurotensin receptor can be monitored with cAMP.

These agonists are ligated to both PVP-polyproline-PVP and large, branched, dextran-PVP (MW˜20,000) polymers. The linear polyproline systems have one agonist bound to each end and the large, branched system has a mixture of both ligands attached to each polymer molecule.

Polymer synthesis and ligation use the building blocks and alkyne-azide cycloaddition reactions shown in Schemes 1-4. The free amine of the adenosine receptor agonist ADAR are conjugated to the PVP-alkyne building block (n=15, 20, and 25) as in Step 4 in Scheme 1. The N-terminus of the neurotensin pentapeptide agonist are linked to the PVP-alkyne building block while on the SPPS resin. This provides ligand-PVP-alkyne building blocks for the molecules in this example.

The polyproline central regions (n=8, 12, 16, and 20) are prepared and linked as in Scheme 3. For the branched multifunctional dextran-PVP polymers, the ligand-PVP-alkyne building blocks are coupled to the azide modified dextran molecules described in Example 4. The ligand-PVP-alkyne building blocks are used as controls in all assays. The compounds tested consist of the 12 combinations of polyproline and PVP building blocks and the three multifunctional dextran compounds prepared with three lengths of PVP linkers.

All molecules are tested for both neurotensin and A2-adenosine receptor activity in cells expressing one or both of these receptors. Cell systems and 96-well assays for these receptors are available commercially. To correct for the possible steric hindrance of the multifunctional linkers, the ligand-PVP-alkyne building blocks are used as controls in all assays. Concentration-activity profiles for the molecules described above are generated for the individual and combined receptor systems. Optimum compounds are then selected based on the resulting values of affinity and activity.

### Example 9

### Synthesis and Testing of Polyproline-Based Bifunctional Ligands

Five polyproline-based bifunctional agonists with rigid central sections spanning 16-80 Å were synthesized and tested with a neurotensin agonist assay. Briefly, molecules were synthesized using solid-phase peptide synthesis (SPPS) to prepare a neurotensin agonist with a Sar5 flexible linker and a terminal azide for conjugation. The agonist building block consisted of an N-terminal 4azidobutyric acid followed by a Sar5 flexible linker and a C-terminal agonist sequence RRPYIL. The agonist building block was purified by reverse phase HPLC and characterized by Mass spectrometry. The polyproline sections had alkyne-functionalized N- and C-termini. The sequences consisted of polyprolines (n=5, 10, 15, 20, and 25) with N- and C-terminal propargyl glycines. Peptides were synthesized using standard SPPs methods and purified by reverse phase HPLC followed by size exclusion chromatography for the Pro20 and Pro25 molecules. The building blocks were characterized by mass spectrometry.

Bifunctional agonists were prepared by combining a polyproline building block with two equivalents of the azide agonist with the alkyne-azide cycloaddition reaction. As a representative reaction, 1.6 mg of the Pro15dialkyne in 63 μL H2O was added to 2.6 mg of the neurotensin agonist azide. To that solution was added 20 μL of a 4.7 mg/mL solution of copper sulfate and a sample was withdrawn for HPLC analysis. Ascorbate (20 μL of a 24 mg/mL solution) was added and samples were withdrawn over the next 45 minutes. The agonist peptide was converted to first two peaks then one peak. An additional 0.2 mg of neurotensin agonist was added to ensure complete conversion to the bifunctional peptide. The compound was isolated by size exclusion HPLC and characterized by mass spectrometry.

Bifunctional polyproline-based neurotensin agonists with rigid central sections of pron (n=5, 10, 15, 20, and 25) were tested in a commercial neurotensin GPCR assay (Invitrogen NTSR1 CHO-K1 DA) according to manufacturer specifications. The observed affinities were 25±4, 28±3, 30±4, 18±2, and 27±2 nM for the Pro5, 10, 15, 20, and 25, respectively. Emax values were 2.6±0.1, 2.60±0.05, 2.7±0.1, 2.66±0.05, and 2.7±0.1 (corrected increase in blue/green fluorescence ratio) for the Pro5, 10, 15, 20, and 25, respectively. This suggests that receptor content was not perturbed. The higher affinity for the Pro20 molecule is consistent with more efficient binding when the appropriate distances (55 Å) have been achieved.

### Example 10

### Synthesis and Testing of a Multifunctional Dextran Molecule

A multifunctional dextran was prepared by alkynylation of 25,000 MW dextran with 4-bromobutyne followed by addition of the azide agonist with the alkyne-azide cycloaddition reaction. Briefly, 15 mg of 25,000 MW dextran was dissolved in 300 μL of water. 0.25 mg of NaBH4 in 25 μL water was added to reduce the aldehyde ends to alcohols. 30 mg of NaOH was added, followed by 60 μL of 4-Bromobutyne. The reaction was stirred overnight at 60° C. The reaction was neutralized with acetic acid and dialyzed against 4×1 L water in 1000 MW cutoff dialysis tubing. The product was lyophilized and weighed giving 15.2 mg product.

To prepare the multifunctional dextran, alkynylated dextran (2.5 mg) was dissolved in 100 μL of 50% water/ethylene glycol. Eight equivalents (1.06 mg) of the neurotensin agonist azide (from Example 1) was added to the reaction, followed by 20 μL of a 4.7 mg/mL solution of copper sulfate. A sample was withdrawn for HPLC analysis. Ascorbate (20 μL of a 24 mg/mL solution) was added, the reaction was heated to 60° C., and the reaction was followed by HPLC-UV at 274 nm. By 2 hours, the agonist peptide peak had disappeared. Since the dextran has a distribution of molecular weights, mass spectral characterization of modified dextrans is not possible. Therefore, incorporation was based on loss of the azide agonist.

The Dex8 molecule (8 ligands per molecule average) was tested in a commercial neurotensin GPCR assay (Invitrogen NTSR1 CHO-K1 DA) according to manufacturer specifications. Dex8 had an affinity of 0.8±2 nM, which is 30-fold higher affinity than the bifunctional polyprolines, respectively. The Emax value was 2.6±0.1, consistent with the bifunctional polyproline molecules. This data suggests that multifunctional dextrans can be used to simultaneously bind to multiple cell surface receptors and that increases in affinity are possible through multivalent interactions.

The present invention is not to be limited in scope by the specific embodiments described above, which are intended as illustrations of aspects of the invention. Functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited references are hereby incorporated by reference.

### REFERENCES

Armstrong, J. K., Wenby, R. B., Meiselman, H. J., & Fisher, T. C. (2004). The hydrodynamic radii of macromolecules and their effect on red blood cell aggregation. Biophysical Journal, 87 (6), 4259-70.
Bhat, R., & Timasheff, S. N. (1992). Steric exclusion is the principal source of the preferential hydration of proteins in the presence of polyethylene glycols. Protein Science: A Publication of the Protein Society, 1 (9), 1133-43.
Bohrer, M. P., Deen, W. M., Robertson, C. R., Troy, J. L., & Brenner, B. M. (1979). Influence of molecular configuration on the passage of macromolecules across the glomerular capillary wall. The Journal of General Physiology, 74 (5), 583-93.
Caliceti, P., & Veronese, F. M. (2003). Pharmacokinetic and biodistribution properties of poly(ethylene glycol)-protein conjugates. Advanced Drug Delivery Reviews, 55 (10), 1261-77.
Cao, Y. (2008). Molecular mechanisms and therapeutic development of angiogenesis inhibitors. Advances in Cancer Research, 100, 113-31.
Collinson, F. J., Hall, G. D., Perren, T. J., & Jayson, G. C. (2008). Development of antiangiogenic agents for ovarian cancer. Expert Review of Anticancer Therapy, 8 (1), 21-32.
Crespo, L., Sanclimens, G., Montaner, B., Pérez-Tomas, R., Royo, M., Pons, M., et al. (2002). Peptide dendrimers based on polyproline helices. Journal of the American Chemical Society, 124 (30), 8876-83.
D'Andrea, L. D., Del Gatto, A., Pedone, C., & Benedetti, E. (2006). Peptide-Based molecules in angiogenesis. Chemical Biology & Drug Design, 67 (2), 115-26.
David, A., Kopecková, P., Minko, T., Rubinstein, A., & Kopecek, J. (2004). Design of a multivalent galactoside ligand for selective targeting of HPMA copolymer-doxorubicin conjugates to human colon cancer cells. European Journal of Cancer (Oxford, England: 1990), 40 (1), 148-57.
Deshayes, S., Morris, M. C., Divita, G., & Heitz, F. (2006). Interactions of amphipathic carrier peptides with membrane components in relation with their ability to deliver therapeutics. Journal of Peptide Science: An Official Publication of the European Peptide Society, 12 (12), 758-65.
Dijkgraaf, I., Rijnders, A. Y., Soede, A., Dechesne, A. C., van Esse, G. W., Brouwer, A. J., et al. (2007). Synthesis of dota-conjugated multivalent cyclic-rgd peptide dendrimers via 1,3-dipolar cycloaddition and their biological evaluation: Implications for tumor targeting and tumor imaging purposes. Organic & Biomolecular Chemistry, 5 (6), 935-44.
Dintzis, R. Z., Okajima, M., Middleton, M. H., & Dintzis, H. M. (1990) Inhibition of antibody formation by receptor cross-linking: The molecular characteristics of inhibitory haptenated polymers. European Journal of Immunology, 20 (1), 229-32.
Dintzis, R. Z., Okajima, M., Middleton, M. H., Greene, G., & Dintzis, H. M. (1989). The immunogenicity of soluble haptenated polymers is determined by molecular mass and hapten valence. Journal of Immunology (Baltimore, Md.: 1950), 143 (4), 1239-44.
Fillon, Y. A., Anderson, J. P., & Chmielewski, J. (2005). Cell penetrating agents based on a polyproline helix scaffold. Journal of the American Chemical Society, 127 (33), 11798-803.
Gauthier, M. A., & Klok, H. A. (2008). Peptide/protein-polymer conjugates: Synthetic strategies and design concepts. Chemical Communications (Cambridge, England), (23), 2591-611.
Geisler, I., & Chmielewski, J. (2007). Probing length effects and mechanism of cell penetrating agents mounted on a polyproline helix scaffold. Bioorganic & Medicinal Chemistry Letters, 17 (10), 2765-8.
Gestwicki, J. E., Cairo, C. W., Strong, L. E., Oetjen, K. A., & Kiessling, L. L. (2002). Influencing receptor-ligand binding mechanisms with multivalent ligand architecture. Journal of the American Chemical Society, 124 (50), 14922-33.
Gonçalves, M., Estieu-Gionnet, K., Berthelot, T., Lain, G., Bayle, M., Canron, X., et al. (2005). Design, synthesis, and evaluation of original carriers for targeting vascular endothelial growth factor receptor interactions. Pharmaceutical Research, 22 (8), 1411-21.
Goodwin, A. P., Tabakman, S. M., Welsher, K., Sherlock, S. P., Prencipe, G., & Dai, H. (2009). Phospholipid-Dextran with a single coupling point: A useful amphiphile for functionalization of nanomaterials. Journal of the American Chemical Society, 131 (1), 289-96.
Greco, F., Vicent, M. J., Gee, S., Jones, A. T., Gee, J., Nicholson, R. I., et al. (2007). Investigating the mechanism of enhanced cytotoxicity of HPMA copolymer-dox-agm in breast cancer cells. Journal of Controlled Release: Official Journal of the Controlled Release Society, 117 (1), 28-39.
Haaf, F., Sanner, A., & Straub, F. (1985). Polymers of n-vinylpyrrolidone: Synthesis, characterization and uses. Polymer Journal, 17 (1), 143-152.
Hammes, G. G., & Schimmel, P. R. (1967). An investigation of water-urea and water-urea-polyethylene glycol interactions 1. Journal of the American Chemical Society, 89 (2), 442-446.
Hodivala-Dilke, K. (2008). Alphavbeta3 integrin and angiogenesis: A moody integrin in a changing environment. Current Opinion in Cell Biology, 20 (5), 514-9.
Hopkins, A. L. (2008). Network pharmacology: The next paradigm in drug discovery. Nature Chemical Biology, 4 (11), 682-90.
Jeppesen, C., Wong, J. Y., Kuhl, T. L., Israelachvili, J. N., Mullah, N., Zalipsky, S., et al. (2001). Impact of polymer tether length on multiple ligand-receptor bond formation. Science, 293 (5529), 465-8.
Kaneda, Y., Tsutsumi, Y., Yoshioka, Y., Kamada, H., Yamamoto, Y., Kodaira, H., et al. (2004). The use of PVP as a polymeric carrier to improve the plasma half-life of drugs. Biomaterials, 25 (16), 3259-66.
Kiessling, L. L., Gestwicki, J. E., & Strong, L. E. (2006). Synthetic multivalent ligands as probes of signal transduction. Angewandte Chemie (International Ed. In English), 45 (15), 2348-68.
Klutz, A. M., Gao, Z. G., Lloyd, J., Shainberg, A., & Jacobson, K. A. (2008). Enhanced A3 adenosine receptor selectivity of multivalent nucleoside-dendrimer conjugates. Journal of Nanobiotechnology, 6, 12.
Kolb, H. C., Finn, M. G., & Sharpless, K. B. (2001). Click chemistry: Diverse chemical function from a few good reactions. Angewandte Chemie (International Ed. In English), 40 (11), 2004-2021.
Kopecek, J., Kopecková, P., Minko, T., & Lu, Z. (2000). HPMA copolymer-anticancer drug conjugates: Design, activity, and mechanism of action. European Journal of Pharmaceutics and Biopharmaceutics: Official Journal of Arbeitsgemeinschaft Für Pharmazeutische Verfahrenstechnik E. V, 50 (1), 61-81.
Langel (2006). Penetratins. In Handbook of cell-penetrating peptides. (pp. 5-28). CRC Press.
Lee, C. C., MacKay, J. A., Fréchet, J. M., & Szoka, F. C. (2005). Designing dendrimers for biological applications. Nature Biotechnology, 23 (12), 1517-26.
Lee, Y., & Sampson, N. S. (2006). Romping the cellular landscape: Linear scaffolds for molecular recognition. Current Opinion in Structural Biology, 16 (4), 544-50.
Livnah, O., Stura, E. A., Middleton, S. A., Johnson, D. L., Jolliffe, L. K., & Wilson, I. A. (1999). Crystallographic evidence for preformed dimers of erythropoietin receptor before ligand activation. Science, 283 (5404), 987-90.
Lu, Z. R., Shiah, J. G., Sakuma, S., Kopecková, P., & Kopecek, J. (2002). Design of novel bioconjugates for targeted drug delivery. Journal of Controlled Release: Official Journal of the Controlled Release Society, 78 (1-3), 165-73.
Luo, Y., Bernshaw, N. J., Lu, Z. R., Kopecek, J., & Prestwich, G. D. (2002). Targeted delivery of doxorubicin by HPMA copolymer-hyaluronan bioconjugates. Pharmaceutical Research, 19 (4), 396-402.
Lutz, J. F., Borner, H. G., & Weichenhan, K. (2006). Combining ATRP and “click” chemistry: A promising platform toward functional biocompatible polymers and polymer bioconjugates. Macromolecules, 39 (19), 6376-6383.
Maeda, H., Bharate, G. Y., & Daruwalla, J. (2008). Polymeric drugs for efficient tumor-targeted drug delivery based on epr-effect. European Journal of Pharmaceutics and Biopharmaceutics: Official Journal of Arbeitsgemeinschaft Für Pharmazeutische Verfahrenstechnik E. V.
Maier, M. A., Esau, C. C., Siwkowski, A. M., Wancewicz, E. V., Albertshofer, K., Kinberger, G. A., et al. (2006). Evaluation of basic amphipathic peptides for cellular delivery of antisense peptide nucleic acids. Journal of Medicinal Chemistry, 49 (8), 2534-42.
Malugin, A., Kopecková, P., & Kopecek, J. (2007). Liberation of doxorubicin from HPMA copolymer conjugate is essential for the induction of cell cycle arrest and nuclear fragmentation in ovarian carcinoma cells. Journal of Controlled Release: Official Journal of the Controlled Release Society, 124 (1-2), 6-10.
Nakatsubo, F., Kamitakahara, H., & Hori, M. (1996). Cationic ring-opening polymerization of 3, 6-di-o-benzyl-[alpha]-d-glucose 1, 2, 4-orthopivalate and the first chemical synthesis of cellulose. J. Am. Chem. Soc, 118 (7), 1677-1681.
Nicolaou, K. C., Estrada, A. A., Zak, M., Lee, S. H., & Safina, B. S. (2005). A mild and selective method for the hydrolysis of esters with trimethyltin hydroxide. Angewandte Chemie (International Ed. In English), 44 (9), 1378-82.
Nishimura, T., & Nakatsubo, F. (1996a). First stepwise synthesis of cellulose analogs. Tetrahedron Letters, 37 (51), 9215-9218.
Nishimura, T., & Nakatsubo, F. (1996b). First synthesis of cellooctaose by a convergent synthetic method. Carbohydrate Research, 294, 53-64.
Nori, A., Jensen, K. D., Tijerina, M., Kopecková, P., & Kopecek, J. (2003). Tat-Conjugated synthetic macromolecules facilitate cytoplasmic drug delivery to human ovarian carcinoma cells. Bioconjugate Chemistry, 14 (1), 44-50.
Ooi, S. L., Pan, X., Peyser, B. D., Ye, P., Meluh, P. B., Yuan, D. S., et al. (2006). Global synthetic-lethality analysis and yeast functional profiling. Trends in Genetics: TIG, 22 (1), 56-63.
Pan, H., Sima, M., Kopecková, P., Wu, K., Gao, S., Liu, J., et al. (2008). Biodistribution and pharmacokinetic studies of bone-targeting N-(2-hydroxypropyl)methacrylamide copolymer-alendronate conjugates. Molecular Pharmaceutics, 5 (4), 548-58.
Penzol, G., Armisén, P., Fernández-Lafuente, R., Rodés, L., & Guisán, J. M. (1998). Use of dextrans as long and hydrophilic spacer arms to improve the performance of immobilized proteins acting on macromolecules. Biotechnology and Bioengineering, 60 (4), 518-23.
Portoghese, P. S. (2001). From models to molecules: Opioid receptor dimers, bivalent ligands, and selective opioid receptor probes. Journal of Medicinal Chemistry, 44 (14), 2259-69.
Portoghese, P. S., Larson, D. L., Sayre, L. M., Yim, C. B., Ronsisvalle, G., Tam, S. W., et al. (1986). Opioid agonist and antagonist bivalent ligands. The relationship between spacer length and selectivity at multiple opioid receptors. Journal of Medicinal Chemistry, 29 (10), 1855-61.
Portoghese, P. S., Ronsisvalle, G., Larson, D. L., & Takemori, A. E. (1986). Synthesis and opioid antagonist potencies of naltrexamine bivalent ligands with conformationally restricted spacers. Journal of Medicinal Chemistry, 29 (9), 1650-3.
Reardon, D. A., Nabors, L. B., Stupp, R., & Mikkelsen, T. (2008). Cilengitide: An integrin-targeting arginine-glycine-aspartic acid peptide with promising activity for glioblastoma multiforme. Expert Opinion on Investigational Drugs, 17 (8), 1225-35.
Rhee, M., & Davis, P. (2006). Mechanism of uptake of C105Y, a novel cell-penetrating peptide. The Journal of Biological Chemistry, 281 (2), 1233-40.
Ryu, J. K., & McLamon, J. G. (2008). VEGF receptor antagonist cyclo-vegi reduces inflammatory reactivity and vascular leakiness and is neuroprotective against acute excitotoxic striatal insult. Journal of Neuroinflammation, 5, 18.
Sanclimens, G., Crespo, L., Giralt, E., Royo, M., & Albericio, F. (2004). Solid-Phase synthesis of second-generation polyproline dendrimers. Biopolymers, 76 (4), 283-97.
Schröder, T., Niemeier, N., Afonin, S., Ulrich, A. S., Krug, H. F., & Brase, S. (2008). Peptoidic amino- and guanidinium-carrier systems: Targeted drug delivery into the cell cytosol or the nucleus. Journal of Medicinal Chemistry, 51 (3), 376-9.
Schuler, B., Lipman, E. A., Steinbach, P. J., Kumke, M., & Eaton, W. A. (2005). Polyproline and the “spectroscopic ruler” revisited with single-molecule fluorescence. Proceedings of the National Academy of Sciences of the United States of America, 102 (8), 2754-9.
Sewald, N., & Jakubke, H. D. (2002). Peptide synthesis. In Peptides: Chemistry and biology. (pp. 135-267). Wiley-VCH.
Smolarczyk, R., Cichoń, T., Graja, K., Hucz, J., Sochanik, A., & Szala, S. (2006). Antitumor effect of RGD-4C-GG-D(KLAKLAK)2 peptide in mouse B16(F10) melanoma model. Acta Biochimica Polonica, 53 (4), 801-5.
Torchilin, V. P., Rammohan, R., Weissig, V., & Levchenko, T. S. (2001). TAT peptide on the surface of liposomes affords their efficient intracellular delivery even at low temperature and in the presence of metabolic inhibitors. Proceedings of the National Academy of Sciences of the United States of America, 98 (15), 8786-91.
Ungar-Waron, H., Gurari, D., Hurwitz, E., & Sela, M. (1973). Role of a rigid polyproline spacer inserted between hapten and carrier in the induction of anti-hapten antibodies and delayed hypersensitivity. European Journal of Immunology, 3(4), 201-5.
Wang, D., Miller, S. C., Liu, X. M., Anderson, B., Wang, X. S., & Goldring, S. R. (2007). Novel dexamethasone-hpma copolymer conjugate and its potential application in treatment of rheumatoid arthritis. Arthritis Research & Therapy, 9 (1), R2.
Weis, S. M., Lindquist, J. N., Barnes, L. A., Lutu-Fuga, K. M., Cui, J., Wood, M. R., et al. (2007). Cooperation between VEGF and beta3 integrin during cardiac vascular development. Blood, 109 (5), 1962-70.
Yano, K., Kimura, S., & Imanishi, Y. (1998). Simultaneous activation of two different receptor systems by enkephalin/neurotensin conjugates having spacer chains of various lengths. European Journal of Pharmaceutical Sciences: Official Journal of the European Federation for Pharmaceutical Sciences, 7 (1), 41-48.
Yoon, Y. R., Lim, Y. B., Lee, E., & Lee, M. (2008). Self-Assembly of a peptide rod-coil: A polyproline rod and a cell-penetrating peptide tat coil. Chemical Communications (Cambridge, England), (16), 1892-4.
Zilberberg, L., Shinkaruk, S., Lequin, O., Rousseau, B., Hagedorn, M., Costa, F., et al. (2003). Structure and inhibitory effects on angiogenesis and tumor development of a new vascular endothelial growth inhibitor. The Journal of Biological Chemistry, 278 (37), 35564-73.

We claim:

1. A multifunctional linker capable of binding a plurality of ligands, having at least one central portion and at least two distal portions, wherein the distal portions are capable of binding ligands.

2. The multifunctional linker of claim 1, wherein the central and distal portions independently comprise a polymer or a derivative thereof.

3. The multifunctional linker of claim 2, wherein the polymer is selected from the group consisting of polysaccharides, hyaluronic acid, PVP, synthetic polymers, and derivatives thereof.

4. The multifunctional linker of claim 3, wherein the polysaccharide is selected from the group consisting of cellulose, dextran, and derivatives thereof.

5. The multifunctional linker of claim 2, wherein either the central portion is rigid and the distal portions are flexible, or the central portion is flexible and the distal portions are rigid.

6. The multifunctional linker of claim 5, wherein the distal portions are rigid and separated from the central portion with flexible hinge regions.

7. The multifunctional linker of claim 1, wherein the central portion is rigid and comprises a polymer having a persistence length of fifteen angstroms or greater, and wherein the distal portions are flexible and comprise a polymer having a persistence lengths of ten angstroms or less.

8. The multifunctional linker of claim 1, wherein its effective length is between 25 and 500 Å.

9. The multifunctional linker of claim 1, wherein its effective length is between 40 and 500 Å.

10. The multifunctional linker of claim 1, wherein its effective length is between 40 and 100 Å

11. The multifunctional linker of claim 1, wherein the distal portions are a rigid polysaccharide separated from the central portion by a flexible linker.

12. The multifunctional linker of claim 1, wherein the distal portions are a rigid helix separated from the central portion by a flexible linker.

13. The multifunctional linker of claim 1, wherein the distal portions are each a rigid synthetic polymer separated from the central portion by a flexible linker.

14. The multifunctional linker of claim 1, wherein the central portion is a helical polymer or a beta sheet.

15. The multifunctional linker of claim 1, wherein the distal portions are natural or synthetic flexible polymers.

16. The multifunctional linker of claim 17, wherein the natural flexible polymer is a polypeptide.

17. The multifunctional linker of claim 1, wherein the distal portions are selected from the group consisting of polyvinylpyrrolidone, polyethyleneglycol (PEG), and polyethyleneoxide (PEO).

18. The multifunctional linker of claim 1, wherein the plurality of distal portions are spaced at desired intervals about the central portion.

19. The multifunctional linker of claim 1, further comprising bound ligands which are each biologically active agents, wherein said ligands are the same or different.

20. A pharmaceutical composition comprising the multifunctional linker with bound ligands of claim 19.
